(19) United States
(12) Patent Application Publication
(10) Pub. No.: US 2019/0035506 A1
(43) Pub. Date: Jan. 31, 2019

(54) INTELLIGENT AUXILIARY DIAGNOSIS METHOD, SYSTEM AND MACHINE-READABLE MEDIUM THEREOF

(71) Applicant: Hefei University of Technology, Hefei (CN)

(72) Inventors: Shuai DING, Hefei (CN); Shanlin YANG, Hefei (CN); Chao FU, Hefei (CN); He LUO, Hefei (CN)

(21) Appl. No.: 16/049,787

(22) Filed: Jul. 30, 2018

(30) Foreign Application Priority Data
Jul. 31, 2017 (CN) 201710642610.5

(51) Int. Cl. G16H 50/20; G16H 10/60; G06N 5/04; G06N 99/00

(52) U.S. Cl. CPC G16H 50/20; G06N 99/005; G06N 5/04; G16H 10/60

(57) ABSTRACT

The invention provides an intelligent auxiliary diagnosis method, system and machine-readable medium. The method comprises: calculating relevancy between keywords of chief complaint in a current medical record and in a standard medical record and Latent Semantic Indexing (LSI) themes to acquire a set of vectors for current medical record-theme relevancy and a set of vectors for standard medical record-theme relevancy; calculating the similarity between the chief complaint in a current medical record and the chief complaint in a standard medical record, based on the set of vectors for current medical record-theme relevancy and the set of vectors for standard medical record-theme relevancy; and determining a corresponding standard medical record, according to the similarity. The invention can be used for preliminary determination of a current medical record and intelligent diagnosis, thereby greatly reducing the pressure on hospital staff and improving patient experience.

### TECHNICAL FIELD

The present invention relates to the field of intelligent medical technologies, and in particular to an intelligent auxiliary diagnosis method, a system and a machine-readable medium thereof.

### BACKGROUND

With the progress of society and the improvement of people's living standard, people pay more attention to their health and people's medical demands are increasing. In addition, some people visit hospital for regular physical examination even without any discomfort.

The traditional disease diagnosis process relies on the doctor's inquiry about the patient's symptoms, and the doctor then makes a decision on the patient's disease according to the answer of the patient and disease features collected by the doctor previously. However, the actual diagnosis process is complex for a patient. A patient must go through a series of flows such as registration, lining up by number and waiting to see the doctor before he/she can finally reach the link of doctor's diagnosis and treatment. In the diagnosis process, a patient needs to line up in each link, and the time for lining up is significantly increased especially in large hospitals. For the whole diagnosis process, the patient may spend averagely two to three hours or even longer on lining up, while the time for actual diagnosis with the doctor may be just ten minutes.

Therefore, for patients, the consulting experience is not pleasant in the traditional disease diagnosis and treatment process. Meanwhile, there is a serious shortage of medical personnel compared with the number of patients and thus the workload of medical personnel is quite heavy.

### SUMMARY

To overcome or at least partially solve the problems, the present invention provides an intelligent auxiliary diagnosis method, a system and a machine-readable medium to implement preliminary determination for the current medical record and intelligent hospitalization guidance, so that the pressure caused by the shortage of medical personnel is greatly mitigated, the workload of medical personnel is reduced and the medical diagnosis experience of patients is improved.

In one aspect, the present invention provides an intelligent auxiliary diagnosis method,comprising steps of: calculating relevancy between a keyword of a chief complaint in a current medical record and Latent Semantic Indexing (LSI) themes to determine a set of vectors for current medical record-theme relevancy; calculating relevancy between a keyword of a chief complaint in a standard medical record and Latent Semantic Indexing (LSI) themes to determine a set of vectors for standard medical record-theme relevancy; calculating similarity between the chief complaint in the current medical record and the chief complaint in the standard medical record, based on the set of vectors for current medical record-theme relevancy and the set of vectors for standard medical record-theme relevancy; and determining a target standard medical record corresponding to the chief complaint in a current medical record according to the similarity.

Wherein, the method further comprises steps of: ranking the determined similarity based on different sets of vectors for standard medical record-theme relevancy, and determining a target standard medical record according to the result of ranking and feedback information based on the standard medical record.

Wherein, the step of determining a target standard medical record according to the result of ranking and feedback information based on the standard medical record, further comprises:

comparing a standard question in each of a plurality of standard medical records to the feedback information based on the standard medical record orderly starting from a standard medical record with the highest similarity, and replacing the standard medical records in sequence based on the comparison of relevancy until the comparison of the ordered standard question in all standard medical records are completed.

Wherein, the step of replacing the standard medical records in sequence based on the comparison of relevancy until the comparison of the ordered standard question in all standard medical records are completed further comprises: selecting a next standard questions in order in the standard medical record, if the comparison of the ordered standard question in each of the plurality of standard medical records, with the feedback information based on the standard medical record fails to meet a set standard.

Wherein, the feedback information based on the standard medical record refers to an answer information acquired from a patient, an answer information of the current medical record feedback or answer information of historical medical record feedback.

Wherein, a standard medical record database comprises a bank of standard medical record chief complaints, a bank of an ordered standard questions, and a bank of standard answer corresponding to the ordered standard question bank.

Further, before the step of calculating relevancy between keywords of chief complaint in a current medical record and LSI themes to acquire a set of vectors for current medical record-theme relevancy, the method further comprises: acquiring the chief complaint in the current medical record and performing word segmentation, stopwords removal and keywords extraction on the chief complaint in the current medical record to acquire a keyword of the chief complaint in the current medical record.

Wherein, acquiring the LSI themes comprises: performing word segmentation and stopwords removal on the chief complaint in the standard medical record to acquire a plurality of words; and classification operating the plurality of words according to the frequency of each of the words appearing in the chief complaint in the standard medical record, to acquire several LSI themes.

Wherein, the step of classification operating the plurality of words according to the frequency of each of the words appearing in the chief complaint in the standard medical record, to acquire several LSI themes comprises: numbering the words according to the sequence numbers of the words in a medical dictionary and calculating the frequency of the words appearing in the chief complaint in the standard medical record; constructing a standard medical record chief complaint document vector containing a pair of the number and the frequency as an element; and calculating a TF-IDF value of the word corresponding to each element in the standard medical record chief complaint document vector to acquire a TF-IDF vector, and acquiring an LSI model by the TF-IDF vector training to set the LSI themes.

In another aspect, the present invention provides an intelligent auxiliary diagnosis system, comprising: one or more non-volatile memories, and a processor, wherein the processor comprises: a first relevancy calculation module, a second relevancy calculation module, a similarity calculation module and a medical record determination module. Wherein, the first relevancy calculation module is configured to calculate relevancy between keywords of chief complaint in a current medical record and LSI themes to determine a set of vectors for current medical record-theme relevancy; the second relevancy calculation module is configured to calculate relevancy between keywords of chief complaint in a standard medical record and the LSI themes to determine a set of vectors for standard medical record-theme relevancy; the similarity calculation module is configured to calculate, based on the set of vectors for current medical record-theme relevancy and the set of vectors for standard medical record-theme relevancy, a similarity between the chief complaint in the current medical record and the chief complaint in the standard medical record; and the medical record determination module is configured to determine, according to the similarity, a target standard medical record corresponding to the chief complaint in a current medical record.

The present invention also provides a machine-readable storage medium executing instructions configured to enable a machine to perform the intelligent auxiliary diagnosis method of the present invention.

The present invention provides an intelligent auxiliary diagnosis method and system and wherein a target standard medical record is determined by gradually matching chief complaint in a current medical record with data in a standard medical record. The target standard medical record can be effectively applied to preliminary determination for the current medical record and intelligent guidance, so that the pressure caused by the shortage of medical personnel is greatly mitigated, the workload of medical personnel is reduced and the medical experience of patients is improved.

### BRIEF DESCRIPTION OF THE DRAWINGS

FIG. 1 is a flowchart of an intelligent auxiliary diagnosis method according to an embodiment of the present invention;

FIG. 2 is a flowchart of a process of acquiring LSI themes according to an embodiment of the present invention;

FIG. 3 is a flowchart of a process of acquiring the LSI themes according to the frequency of words according to an embodiment of the present invention;

FIG. 4 is a schematic diagram of a standard medical record database according to an embodiment of the present invention;

FIG. 5 is a schematic diagram of an intelligent auxiliary diagnosis system according to an embodiment of the present invention; and

FIG. 6 is a schematic diagram of hardware implementation of an intelligent auxiliary diagnosis system according to an embodiment of the present invention.

### DETAILED DESCRIPTION

To make the objectives, technical solutions and advantages of the present invention clearer, the technical solutions in the present invention will be described clearly and completely with reference to the drawings in the embodiments of the present invention. Apparently, the described embodiments are just some but not all of the present invention. All other embodiments acquired by those of ordinary skill in the art without making any creative effort shall fall into the protection scope of the present invention.

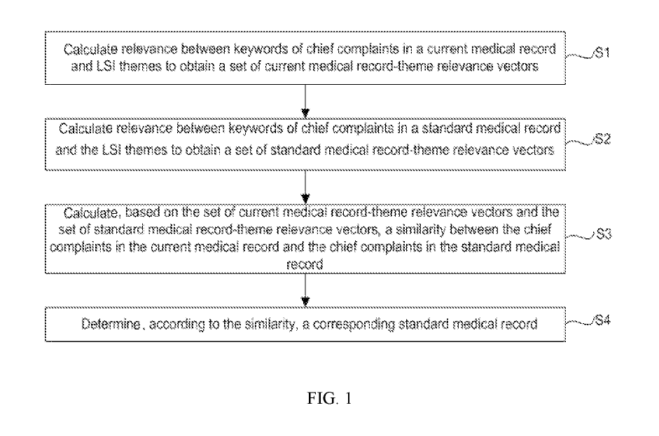

As one aspect of the embodiment of the present invention, this embodiment provides an intelligent auxiliary diagnosis method executable by a computer. FIG. 1 provides a flowchart of an intelligent auxiliary diagnosis method according to an embodiment of the present invention. The method comprises the following steps:

S1: calculating relevancy between keywords of chief complaint in a current medical record and LSI themes to acquire a set of vectors for current medical record-theme relevancy;

S2: calculating relevancy between keywords of chief complaint in a standard medical record and the LSI themes to acquire a set of vectors for standard medical record-theme relevancy;

S3: calculating similarity between the chief complaint in the current medical record and the chief complaint in the standard medical record, based on the set of vectors for current medical record-theme relevancy and the set of vectors for standard medical record-theme relevancy; and

S4: determining a corresponding standard medical record according to the similarity,

Prior to the specific description of the steps S1 and S2, several definitions will be given as follows.

Chief complaint in a medical record: a medical and psychological term. The subject of the medical record states the main suffering he/she has, the main reason for diagnosis or the most obvious symptoms, signs and/or nature, and the duration of these symptoms, which preliminarily reflects the disease severity and provides diagnosis clues for a certain system disease. The chief complaint in a medical record needs to be accurate, objective and practical. For example, a good chief complaint should be accurate, and the symptoms described by the subject of the medical record should be consistent with the history of present illness of the subject of the medical record.

Keywords of chief complaint in a medical record: The chief complaint in a medical record is generally a paragraph of description in natural language. Several keywords which can completely express the meaning of chief complaint of the subject of the medical record may be extracted by performing certain processing on the chief complaint in the medical record. The keywords are regarded as the keywords of chief complaint in a medical record. In the following description, “several” refers to “one or more”.

Latent Semantic Indexing (LSI) model: a natural language processing model, by which the relation between words is found from massive literatures. When two words or a set of words appear in a document frequently, these words may be considered semantically related. Related words are found by the statistical process of massive data to constitute a latent theme. Essentially, it is word clustering. The LSI model in the embodiment of the present invention is an LSI model for chief complaint in a standard medical record, which is established by statistical process and clustering of standard medical record data.

LSI themes: several latent themes constituted by related words which are acquired by statistical process and clustering of standard medical record chief complaint data.

Chief complaint in a standard medical record: The standard medical record database stores standard medical record data corresponding to diseases, comprising chief complaint information of these standard medical records. The patient of the standard medical record states the main suffering he/she has, the main reason for diagnosis or the most obvious symptoms, signs and/or nature, and the duration of these symptoms, which preliminarily reflects the disease severity and provides diagnosis clues for a certain system disease.

Keywords of chief complaint in a standard medical record: The chief complaint in a standard medical record is generally a paragraph of description in natural language. Several keywords which can completely express the meaning of chief complaint of the patient of the standard medical record may be extracted by performing certain processing on the chief complaint in the standard medical record. The keywords are the keywords of chief complaint in a standard medical record.

For the steps S1 and S2, specifically, after keywords are extracted from the standard medical record chief complaint data and the statistical process and clustering of the keywords are completed, several LSI themes are set according to the clustering information and the LSI model is established and trained. Meanwhile, natural language processing is performed on the chief complaint in the current medical record and the chief complaint in the standard medical record, respectively, and keywords of the chief complaint in the current medical record and keywords of the chief complaint in the standard medical record are extracted, respectively.

Then, relevancy between the keywords of the chief complaint in the current medical record and the LSI themes is calculated by the trained LSI model to acquire a set of vectors for current medical record-theme relevancy, and relevancy between the keywords of the chief complaint in the standard medical record and the LSI themes is calculated by the trained LSI model to acquire a set of vectors for standard medical record-theme relevancy.

In one embodiment, before the step of calculating relevancy between keywords of chief complaint in a current medical record and LSI themes to acquire a set of vectors for current medical record-theme relevancy, the method further comprises: acquiring the chief complaint in the current medical record and performing word segmentation, stopwords removal and keyword extraction on the chief complaint in the current medical record to acquire the keywords of the chief complaint in the current medical record.

Similarly, before the step of calculating relevancy between keywords of chief complaint in a standard medical record and LSI themes to acquire a set of vectors for standard medical record-theme relevancy, the method further comprises: acquiring the chief complaint in the standard medical record and performing word segmentation, stopwords removal and keyword extraction on the chief complaint in the standard medical record to acquire the keywords of the chief complaint in the standard medical record.

Wherein, word segmentation refers to segmenting a sequence of Chinese characters into individual words. That is, it is a process of recombining a sequence of successive characters into a sequence of words according to a certain criterion. There are three existing word segmentation methods: word segmentation based on string matching, word segmentation based on understanding, and word segmentation based on statistical process. There are other two word segmentation methods, i.e., simple word segmentation and word segmentation combined with tagging, depending upon whether the word segmentation is performed together with a part-of-speech tagging process.

stopwords removal refers to removing part of words in a paragraph which have no or little effect on the main meaning of the paragraph. These words may appear frequently in the paragraph, but have no effect on the meaning expressed by the paragraph, for example, Chinese auxiliary words such as “de ( [symbol] )”, “de ( [symbol] )” and “de ( [symbol] )”, interjections such as “ah ( [symbol] )”, “ha ( [symbol] )” and “oh ( [symbol] )”, and adverbs or prepositions such as “thereby ( [symbol] )”, “with ( [symbol] )” and “however ( [symbol] )”.

Specifically, voice chief complaint information in the current medical record is recognized by a voice recognition unit, and the voice chief complaint information is converted into text information; or, input text information in the current medical record is acquired directly by a text typing module. The text information is used as current medical record chief complaint information, and the current medical record chief complaint information is used as input to subsequent calculation steps.

Then, word segmentation and stopwords removal are performed on the chief complaint in the current medical record to extract the keywords of the chief complaint in the current medical record and thus to acquire a set of keywords of the chief complaint in the current medical record. relevancy between each keyword of the chief complaint in the current medical record in the set of keywords and M LSI themes is calculated by the LSI model to acquire a set of vectors for current medical record-theme relevancy, respectively. That is, for any one of the keywords of the chief complaint in the current medical record, the following may be acquired:

Patient=[(0, rel₀), (1, rel₁), . . . , (M−2, rel_{M-2}), (M−1, rel_{M-1}),];

Where, the vector Patient represents a current medical record-theme relevancy vector corresponding to any one of the keywords of the chief complaint in the current medical record, 0, 1, 2, . . . , M−1 represent the serial number of M LSI themes; relo, rel₁, rel₂, . . . , rel_{M-1} represent relevancy between the keywords of the chief complaint in the current medical record and the LSI themes numbered from 0 to M−1, respectively.

Meanwhile, the standard medical record chief complaint information is acquired from the standard medical record database, and word segmentation and stopwords removal are performed on the acquired chief complaint in the standard medical record to extract the keywords of the chief complaint in the standard medical record. For any one of the keywords of the chief complaint in the standard medical record, relevancy between the keyword of the chief complaint in the standard medical record and M LSI themes is calculated by the LSI model to acquire standard medical record—theme relevancy vectors corresponding to the keyword of the chief complaint in the standard medical record and establish related indexes, respectively. Wherein, the standard medical record—theme relevancy vectors are expressed by:

EMRₙ=[(0, rel₀′), (1, rel₁′), . . . , (M−2, rel_{M-2}′), (M−1, rel_{M-1}′)]

where, the vector EMRₙ represents a standard medical record-theme relevancy vector corresponding to any one of the keywords of the chief complaint in the standard medical record, 0, 1, 2, . . . , M−1 represent the serial number of M LSI themes; rel₀′, rel₁′, . . . , rel_{M-}1′ represent relevancy between the keywords of the chief complaint in the current medical record and the LSI themes numbered from 0 to M−1, respectively.

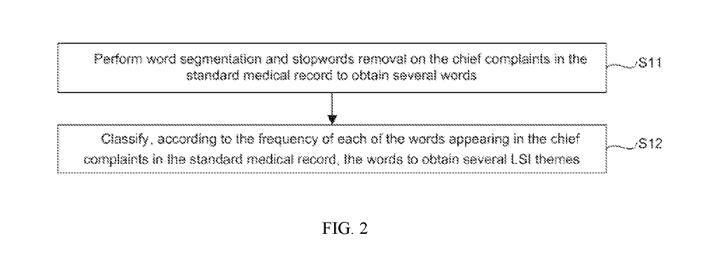

Optionally, the process of acquiring the LSI themes refers to FIG. 2. FIG. 2 is a flowchart of a process of acquiring the LSI themes according to an embodiment of the present invention. The process comprises the following steps.

S11: Word segmentation and stopwords removal are performed on the chief complaint in the standard medical record to acquire several words.

Specifically, before an LSI model is used for calculation, the LSI model needs to be established and be trained by the standard medical record chief complaint information to acquire the LSI themes set during the establishment of the LSI model. That is, for any standard medical record in the standard medical record database, first, corresponding text information of chief complaint in the standard medical record is acquired, and then word segmentation and stopwords removal as described in this embodiment are performed on the text information to acquire several words of the text information of chief complaint in the standard medical record.

S12: The words are classified according to the frequency of each of the words appearing in the chief complaint in the standard medical record to acquire several LSI themes.

Specifically, after the step of acquiring any one of the words of the chief complaint in the standard medical record, a TF-IDF value of each word is calculated by calculating the frequency of the word appearing in the chief complaint in the standard medical record, all words in the standard medical record are classified according to the TF-IDF value, and M themes are set according to the classification information.

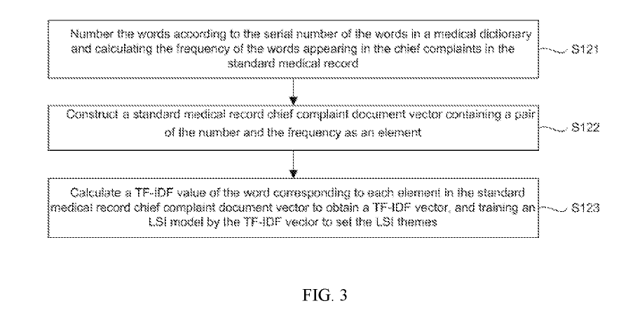

Optionally, the process of classfication operation, according to the frequency of each of the words appearing in the chief complaint in the standard medical record, the words to acquire several LSI themes refers to FIG. 3. FIG. 3 is a flowchart of a process of acquiring the LSI themes according to an embodiment of the present invention. The process comprises the following steps.

S121: The words are numbered according to the sequence numbers of the words in a medical dictionary and the frequency of the words appearing in the chief complaint in the standard medical record is calculated.

Specifically, a medical dictionary needs to be established in advance according to all standard medical record chief complaint information. That is, chief complaint information in all standard medical record base tables in the database is extracted, word segmentation and stopwords removal as described in this embodiment are performed on the chief complaint information to acquire a series of words, the total frequency of each word appearing in all standard medical record chief complaint is calculated, medical related texts with the total frequency exceeding a set threshold are selected, and the selected medical related texts are ranked and numbered to constitute a medical dictionary.

For the established medical dictionary, word segmentation and stopwords removal are performed on the text information of chief complaint in any standard medical record to acquire a set of words of the text information of chief complaint. Each word in the set of words is numbered according to its position number appearing in the medical dictionary, and its frequency num, appearing in the chief complaint is calculated.

S122: A standard medical record chief complaint document vector containing a pair of the number and the frequency as an element is constructed.

Specifically, for the serial number of any one of the words of the chief complaint in the standard medical record and its frequency appearing in the chief complaint in the standard medical record acquired in the above step, the chief complaint are represented as document vectors [id, [(num₀, id₀), (num₁, id₁), . . . , (numₙ, idₙ), . . . , (num_{N}, id_{N})]] where id is used as the primary key. Where, idₙ is the serial number of the set of words divided from the chief complaint in the medical dictionary.

S123: A TF-IDF value of the word corresponding to each element in the standard medical record chief complaint document vector is calculated to acquire a TF-IDF vector, and an LSI model is trained by the TF-IDF vector to set the LSI themes.

Specifically, TF-IDF values tfidfn of words are calculated based on the document vectors in the above step, and new tfidf vectors [id, [(num0, tfidf1), (num1, tfidf2), . . . , (numn, tfidfn), . . . , (numN, tfidfN)]] are generated according to these TF-IDF values. M themes are set according to the tfidf vectors. In this case, documents are expressed by vectors which are represented by the TF-IDF values, and the LSI model is trained by these vectors.

In the step S3, the similarity may be calculated by cosine similarity calculation or Pearson similarity calculation. The following description will be given by using the cosine similarity calculation as example. The cosine similarity calculation refers to evaluating the similarity between two vectors by calculating the cosine value of the comprised angle between the two vectors. Generally, the process of the cosine similarity calculation is as follows: two vectors are drawn in a vector space (for example, the most common 2D space) according to their coordinates; and the comprised angle between the two vectors is acquired and the cosine value of the comprised value is acquired. The cosine value represents the similarity between the two vectors. A smaller comprised angle between the two vectors has a cosine value closer to 1. The directions of the two vectors are more consistent. It means that the two vectors are more similar.

In the step S3, specifically, for any one of vectors in the set Patient of current medical record—theme relevancy vectors and any one of vectors in the set EMRₙ of standard medical record—theme relevancy vectors, the cosine value of the comprised angle between two vectors is calculated according to the coordinates of the two vectors and the similarity between the two vectors is judged according to the calculated cosine value. A larger cosine value indicates a higher similarity between corresponding two vectors. It means that the chief complaint in the current medical record is closer to the standard medical record type corresponding to the standard medical record—theme relevancy vector in the two vectors.

In the step S4, specifically, there is a standard question bank corresponding to each type of standard medical records in the standard medical record database. Standard questions in the standard question bank are ranked in sequence to form ordered standard questions. The ordered standard questions are questions about medical history of the patient in the corresponding standard medical record. For the similarity between the chief complaint in the current medical record and the chief complaint in the standard medical record acquired in the above step, a standard medical record with high similarity to the chief complaint in the current medical record (i.e. a standard medical record with high similarity) is selected according to the degree of similarity. There are several standard questions in a question bank corresponding to the standard medical record with high similarity. After the standard medical record with high similarity is selected, a corresponding standard question bank is accessed, and standard questions in the corresponding standard question bank are compared with feedbacks from the current medical record.

By the intelligent auxiliary diagnosis method according to the embodiment of the present invention, a target standard medical record is determined by calculating and matching chief complaint in a current medical record and chief complaint in a standard medical record. The target standard medical record can be effectively applied to preliminary determination for the current medical record and intelligent guidance, so that the pressure caused by the shortage of medical personnel is greatly mitigated, the workload of medical personnel is reduced and the medical experience of patients is improved.

Optionally, the method further comprises steps of: ranking the acquired similarity, based on different sets of vectors for standard medical record-theme relevancy; and determining a target standard medical record, according to the result of ranking and feedback information based on the standard medical record.

Specifically, in this embodiment, after the similarity between the chief complaint in the current medical record and the chief complaint in each standard medical record are acquired by cosine similarity calculation or other algorithms, corresponding standard medical records are ranked in sequence of corresponding similarity from the high to the lowest. That is, standard medical records corresponding to chief complaint in the standard medical records with high similarity to the chief complaint in the current medical record are ranked in the front, followed by standard medical records corresponding to chief complaint in the standard medical records with low similarity to the chief complaint in the current medical record.

Then, starting from the first standard medical record, answers in the standard medical record are compared with the feedbacks from the current medical record one by one according to questions in each standard medical record question bank. That is, answers to questions in the first standard medical record are first compared with the feedbacks from the current medical record, answers to questions in the second standard medical record are compared with the feedbacks from the current medical record, answers to questions in the third standard medical record are compared with the feedbacks from the current medical record, and so on, until the feedbacks from the current medical record to ordered standard questions in a certain standard medical record meet set conditions. Then, the standard medical record is output as the target standard medical record.

Optionally, the feedback information based on the standard medical record refers to the acquired patient answer information, answer information of the current medical record feedback or answer information of historical medical record feedback. Specifically, the feedback information based on the standard medical record may be one of patient answer information, answer information of the current medical record feedback or answer information of historical medical record feedback or a combination thereof.

Optionally, the step of determining a target standard medical record, according to the result of ranking and feedback information based on the standard medical record, further comprises: comparing, starting from a standard medical record with the highest similarity, ordered standard questions in each standard medical record from beginning to end, with the feedback information based on the standard medical record, and replacing the standard medical records in sequence based on the comparison of relevancy until the comparison of ordered standard questions in all standard medical records are completed.

Optionally, the step of replacing the standard medical records in sequence based on the comparison of relevancy until the comparison of ordered standard questions in all standard medical records are completed further comprises: selecting, if the result of comparison of the ordered standard questions in each of the standard medical records from front to back with the feedback information based on the standard medical record cannot meet a set standard, ordered standard questions in the next standard medical record in sequence.

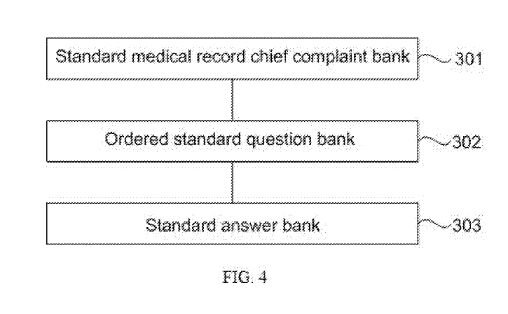

For all standard medical records, there is one standard medical record database. In one embodiment, the structure of the standard medical record database refers to FIG. 4. FIG. 4 is a schematic diagram of a standard medical record database according to an embodiment of the present invention. The standard medical record database comprises a standard medical record chief complaint bank 301, an ordered standard question bank 302, and a standard answer bank 303 corresponding to the ordered standard question bank.

In the embodiment, specifically, first, a standard answer to each question in the question bank of the first standard medical record is compared with the feedbacks from the current medical record. It is judged whether the feedbacks from the current medical record and the answer to the question in the standard medical record database reach a certain relevancy threshold. For example, it is judged whether the first relevancy between the feedbacks from the current medical record and an answer to a same question in the standard medical record database meets a set standard. If it can meet the set standard, a standard answer to the next question is selected to be compared with the feedbacks from the current medical record. If an answer in the current medical record to a certain question in the first standard medical record doesn't meet the set standard, the second standard medical record is selected, i.e. the standard medical record with the second-highest similarity.

After the standard medical record with the second-highest similarity is selected in the above step, a standard answer to each question in the question bank of the second standard medical record is compared with the feedbacks from the current medical record. That is, starting from the first question in the standard medical record with the second-highest similarity, the standard answer to each question in the standard medical record is compared with the feedbacks from the current medical record to the question one by one, relevancy between the patient's answer and the answer to the question in the standard medical record database is calculated to acquire a second relevancy, and the feedbacks from the current medical record are evaluated according to the second relevancy, for example, whether the relevancy between the feedbacks from the current medical record and the answer to the question in the standard medical record database meets the set standard.

Each standard medical record in the standard medical record database corresponds to one ordered question bank. In the above embodiment, standard medical records are ranked according to the similarity to the current medical record chief complaint information, wherein the first one is the standard medical record with the highest similarity. First, a standard answer to the first question in the standard medical record with the highest similarity is selected to be compared with the feedbacks from the current medical record to the question, and relevancy between the both is calculated as a first answer relevancy.

Optionally, the feedbacks from the current medical record further comprise an answer bank for the current medical record or on-site answers in the current medical record. After the step of selecting ordered standard questions in each standard medical record, the method further comprises: judging whether a selected question is in the question and answer bank of the current medical record and acquiring an answer to the question in the current medical record from the question and answer bank of the current medical record if the selected question is in the question and answer bank of the current medical record; and collecting on-site answer in the current medical record if the selected question is not in the question and answer bank of the current medical record and storing the on-site answer in the question and answer bank of the current medical record.

Specifically, the answer to the question in the current medical record may be acquired by collecting the on-site answer in the current medical record in real time. If there is the answer to the question in the historical medical record of the current medical record, the answer to the question in the current medical record may be extracted from the question and answer bank of the historical medical record data of the current medical record.

In the embodiment, after a question in the standard medical record is selected, first, the historical medical record data of the current medical record is searched, it is judged whether the question is asked in the current medical record, and it is judged whether the current medical record answers the question, that is, it is judged whether there is answer data to the question in the historical medical record data of the current medical record. If it is known by search and judgment that there is an answer to the question in the current medical record in the historical medical record data of the current medical record, the answer data is directly read from the current medical record data.

On the other hand, if the historical medical record data of the current medical record shows that the question is not asked in the current medical record, or the question is asked but there is no answer to the question in the current medical record, that is, there is no answer data to the question in the current medical record data, the question is asked in the current medical record and the on-site answer in the current medical record is presented. After the current medical record answers the question on site and the system collects on-site answer data of the current medical record, the system stores the on-site answer data of the current medical record in the question and answer bank of the current medical record.

Then, after the first answer relevancy between the answer in the current medical record to the first question in the standard medical record with the highest similarity and the answer to the question in the standard medical record database is acquired, the relevancy is compared with the set standard. If the relevancy meets the set standard, the second question in the standard medical record with the highest similarity is selected.

According to the above step, the historical medical record database of the current medical record may be searched after the second question is selected, and it is judged whether the current medical record answers the second question, that is, it is judged whether there is an answer to the second question in the current medical record in the historical medical record database of the current medical record. If there is an answer, the answer is directly read; if there is no answer, the second question is asked in the current medical record and an on-site answer to the second question in the current medical record is acquired. relevancy between the answer in the current medical record and answer data to the question in the standard medical record database is calculated according to the answer to the second question in the current medical record, and the relevancy is the next answer relevancy.

And then, the next answer relevancy is compared with the set standard, and it is judged whether the next answer relevancy meets the set standard. If it meets the set standard, the next question in the standard medical record is selected in sequence. The operation is repeated, until the last question in the standard medical record is asked. If the symptoms in the current medical record are highly similar to the symptoms in the standard medical record, then the output diagnosis result is the closest standard medical record.

Or, in the above questioning and answering step, when the questions in the standard medical record with the highest similarity are answered, if the relevancy between an answer to a certain question in the current medical record and an answer to the question in the standard medical record database cannot meet the set standard, it means that the symptoms in the current medical record differ from the symptoms in the standard medical record with the highest similarity. Therefore, the question bank of the next standard medical record (i.e. the standard medical record with the second-highest similarity) is selected according to the ranking of similarity in the above embodiment, and the first question is asked for the current medical record according to the ranking of questions in the question bank. Wherein, the asking process is similar to the asking process for the standard medical record with the highest similarity. Similar operation is performed by taking this process as a rule, until a standard medical record which is most similar to the current medical record data is found. Then, as the diagnosis result, the closest standard medical record is output.

In the intelligent auxiliary diagnosis method according to the embodiment of the present invention, the strict standard of clinical thinking paths is ensured by a sufficient number of medical records which have been verified by specialists and which accord with the clinical thinking paths, and meanwhile, a standard medical record which is closest to the current medical record is found by fuzzy matching to determine a target standard medical record.

In sequence to describe the present invention more clearly, by taking the on-site answer in the current medical record for example, the complete flow according to the embodiment will be described below.

Step 1: Chief complaint information in a standard medical record base table in the database is extracted, and word segmentation and stopwords removal are performed on the chief complaint information to acquire a series of words, and then the frequency of each word is calculated and medical related texts with the frequency exceeding a certain threshold are selected to constitute a medical dictionary.

Word segmentation and stopwords removal are performed on any text information of chief complaint to acquire a set of words of the text information of chief complaint, and each word is numbered. The frequency numn of the word in the chief complaint is calculated, and the chief complaint are expressed by document vectors [id, [(num₀, id₀), (num₁, id₁), . . . , (numₙ, idₙ), . . . , (num_{N}, id_{N})]] where id is used as the primary key. Wherein, idₙ is the serial number of the set of words divided from the chief complaint in the medical dictionary.

TF-IDF values tfidfn of words are calculated based on the document vectors, new tfidf vectors [id, [(num₀, tfidf₁), (num₁, tfidf₂), . . . , (numₙ, tfidfₙ), . . . , (num_{N}, tfidf_{N})]] are generated, and M themes are set. In this case, documents are expressed by vectors which are represented by the TF-IDF values, and the LSI model is trained by these vectors.

Step 2: The chief complaint in the current medical record are acquired in voice or text, and word segmentation, stopwords removal and keyword extraction are performed on the chief complaint in the current medical record to acquire a set of keywords of the chief complaint in text. A set of relevancy vectors between the keywords of the chief complaint in the current medical record and the LSI themes is calculated by the LSI model:

Patient=[(0,rel₀), (1,rel₁), . . . , (M−2, rel_{M-2}), (M−1, rel_{M-1}),],

where, 0, 1, 2, . . . , M−1 represent the serial number of M LSI themes; rel₀, rel₁, rel₂, . . . , rel_{M-}1 represent relevancy between the keywords of the chief complaint in the current medical record and the LSI themes numbered from 0 to M−1, respectively.

Step 3: Word segmentation, stopwords removal and keyword extraction are performed on the chief complaint in the standard medical records in the database, and a set of relevancy vectors between the set of keywords and the themes is calculated by the LSI model:

EMRₙ=[(0, rel₀′), (1, rel₁′), . . . , (M−2, rel_{M-2}′), (M−1, rel_{M-1}′)],

where, 0, 1, 2, . . . , M−1 represent the serial number of M LSI themes; rel₀, rel₁, . . . , rel_{M-}1 represent relevancy between the keywords of the chief complaint in the current medical record and the LSI themes numbered from 0 to M−1, respectively.

Step 4: Cosine similarity calculation is performed on the chief complaint in the current medical record and the chief complaint in the standard medical records by the set of vectors Patient and EMRₙ, and the standard medical records are ranked intelligently according to the result of similarity calculation.

Step 5: A standard medical record with the highest similarity to the chief complaint in the current medical record in the database is selected, and the first question in the standard medical record is asked for the current medical record.

Step 6: It is judged whether the question exists in the question and answer bank of the current medical record. If the question exists, an answer of the current medical record in the bank is extracted and the process proceeds to step 7; if the question does not exist, the current medical record gives a corresponding answer to the question in voice or text, the corresponding question and the answer of the current medical record are stored in the question and answer bank of the current medical record, and the process proceeds to step 7.

Step 7: stopwords removal is performed on the answer of answer in the current medical record and the flow refers to the step 1. Fuzzy matching is performed and the relevancy is calculated. If the relevancy reaches a corresponding relevancy, the next question in the standard medical record is asked and the process proceeds to the step 6; if the relevancy doesn't meet the corresponding requirement, the next standard medical record is selected according to the result of chief complaint similarity ranking in the step 4 and the process proceeds to the step 6.

Step 8: If the asked question is the last question in the standard medical record, the standard medical record is determined as the target standard medical record.

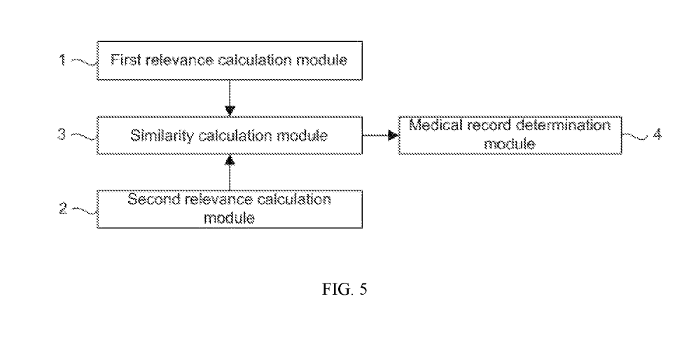

As another aspect of the embodiment of the present invention, the embodiment provides an intelligent auxiliary diagnosis system. FIG. 5 is a schematic diagram of an intelligent auxiliary diagnosis system according to an embodiment of the present invention. The system comprises a first relevancy calculation module 1, a second relevancy calculation module 2, a similarity calculation module 3 and a medical record determination module 4.

Wherein, the first relevancy calculation module 1 is configured to calculate relevancy between keywords of chief complaint in a current medical record and LSI themes to acquire a set of vectors for current medical record-theme relevancy; the second relevancy calculation module 2 is configured to calculate relevancy between keywords of chief complaint in a standard medical record and the LSI themes to acquire a set of vectors for standard medical record-theme relevancy; the similarity calculation module 3 is configured to calculate, based on the set of vectors for current medical record-theme relevancy and the set of vectors for standard medical record-theme relevancy, a similarity between the chief complaint in the current medical record and the chief complaint in the standard medical record; and the medical record determination module 4 is configured to determine, according to the similarity, a corresponding standard medical record.

Specifically, after keyword extraction is performed on the standard medical record chief complaint data and the statistical process and clustering of the keywords are completed, the first relevancy calculation module 1 sets several LSI themes according to the clustering information, and establishes and trains the LSI model. Meanwhile, the first relevancy calculation module 1 and the second relevancy calculation module 2 perform natural language processing on the chief complaint in the current medical record and the chief complaint in the standard medical record, respectively, and extract the keywords of the chief complaint in the current medical record and the keywords of the chief complaint in the standard medical record, respectively.

Then, by the trained LSI model, the first relevancy calculation module 1 calculates the relevancy between the keywords of the chief complaint in the current medical record and the LSI themes to acquire a set of vectors for current medical record-theme relevancy; and the second relevancy calculation module 2 calculates the relevancy between the keywords of the chief complaint in the standard medical record and the LSI themes to acquire a set of vectors for standard medical record-theme relevancy.

For any one of vectors in the set of current medical record—theme relevancy vectors and any one of vectors in the set of standard medical record—theme relevancy vectors, the similarity calculation module 3 calculates the cosine value of the comprised angle between two vectors according to the coordinates of the two vectors and judges the similarity between the two vectors according to the calculated cosine value. A larger cosine value indicates a higher similarity between corresponding two vectors. It means that the chief complaint in the current medical record is closer to the standard medical record type corresponding to the standard medical record—theme relevancy vector in the two vectors.

In addition, there is a standard question bank corresponding to each type of standard medical records in the standard medical record database, and questions in the question bank are ranked in sequence. The similarity calculation module 3 calculates the similarity between the acquired chief complaint in the current medical record and the chief complaint in the standard medical records, and the medical record determination module 4 selects a standard medical record with high similarity to the chief complaint in the current medical record (i.e. a standard medical record with high similarity), according to the degree of similarity. There are several standard questions in a question bank corresponding to the standard medical record with high similarity. After the standard medical record with high similarity is selected, the medical record determination module 4 accesses the corresponding question bank and compares answers to standard questions therein with the feedbacks to the questions in the current medical record.

Beneficial effects of the intelligent auxiliary diagnosis system according to the embodiment of the present invention are the same as those of the method embodiment described above, and thus may refer to the method embodiment described above and will not be repeated here.

Further, the system further comprises a clinical thinking training management module configured to connect to the database and access and manage standard medical record data and current medical record data in the database.

Specifically, the clinical thinking training management module is connected to the database, and it may access and manage the medical record data in the database, including the standard medical record data and all current medical record data. The medical record data comprises chief complaint data, ordered question data of the standard medical record, answer data to questions in the standard medical record, and feedback data from the current medical record. Medical record types and questions corresponding to the medical record types in the standard medical record data are ranked.

The diagnosis system may access data in the database and manage and maintain user data in the database by the clinical thinking training management module.

In the intelligent auxiliary diagnosis system according to the embodiment of the present invention, the database is accessed, managed and maintained by providing the clinical thinking training management module, the reliability of the diagnosis is improved, and the service life of the diagnosis system is prolonged.

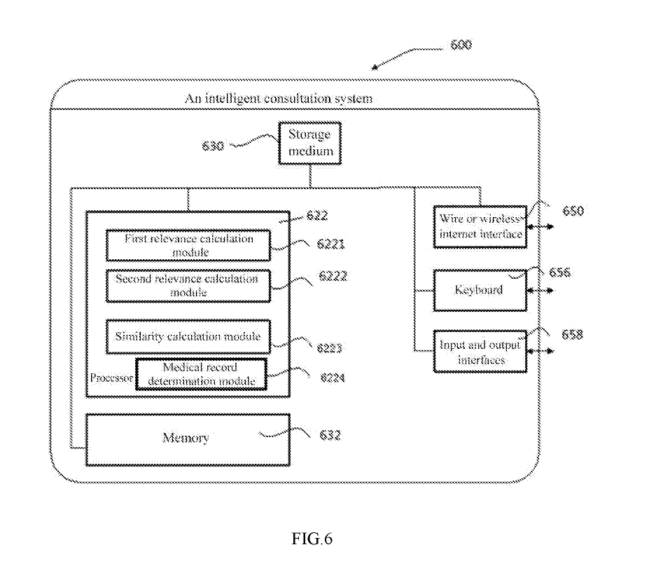

FIG. 6 is a schematic diagram of implementation of the hardware of the intelligent auxiliary diagnosis system according to an embodiment of the present invention. The system 600 can vary dramatically depending on its configuration or performance, and it may comprise one or more central processing units (CPUs) 622 (eg, one or more processors) and a memory 632, one or more storage applications or storage medium 630 of data (for example, one or more mass storage device).Wherein, the memory 632 and the storage medium 630 may be transient storage or permanent storage. Further, CPU 622 can be configured to communicate with storage medium 630 and to perform a series of instructions and operations in storage medium 630 in system 600.

For example, CPU 622 comprises a first relevancy calculation module 6221, a second relevancy calculation module 6222, a similarity calculation module 6223, and a diagnosis processing module 6224.

The first relevancy calculation module 6221 may calculate relevancy between keywords of chief complaint in a current medical record and LSI themes to determine a set of vectors for current medical record-theme relevancy.

The second relevancy calculation module 6222 may calculate relevancy between keywords of chief complaint in a standard medical record and LSI themes to determine a set of vectors for standard medical record-theme relevancy

The similarity calculation module 6223 may calculate the similarity between chief complaint in a current medical record and chief complaint in a standard medical record based on a set of vectors for current medical record-theme relevancy and a set of vectors for standard medical record-theme relevancy.

The diagnosis processing module 6224 may determine a target standard case corresponding to chief complaint in a current medical record according to the similarity.

In some embodiments, CPU 622 can be further configured to acquire the LSI theme by the steps below: performing word segmentation and stopwords removal and keyword extraction on the chief complaint in the standard medical record to acquire several words; classification operating the words to acquire several LSI themes, according to the frequency of each of the words appearing in the standard medical record.

In some embodiments, CPU 622 can be configured to acquire the chief complaint in the current medical record, perform word segmentation, stopwords removal and keyword extraction on the chief complaint in the current medical record to acquire the keywords of the chief complaint in the current medical record.

In some embodiments, CPU 622 can be configured to acquire the chief complaint in the standard medical record, and to perform word segmentation, stopwords removal and keyword extraction on the chief complaint in the current medical record to acquire the keywords of the chief complaint in the standard medical record.

In some embodiments, CPU 622 may comprise a digital signal processor (DSP) which can be configured to calculate relevancy between keywords of chief complaint in a current medical record and Latent Semantic Indexing (LSI) themes to determine a set of vectors for current medical record-theme relevancy, and calculate relevancy between keywords of chief complaint in a standard medical record and Latent Semantic Indexing (LSI) themes to determine a set of vectors for standard medical record-theme relevancy.

The storage medium 630 can store various data required by the intelligent auxiliary diagnosis system 600. For example, in one exemplary embodiment, storage medium 630 can store a chief complaint in a medical record acquired from a medical record subject. And wherein the chief complaint in a medical record can be stored in any form or structure by those of skilled in the art. In such an embodiment, CPU 622 can acquire chief complaint in a medical record from the storage medium 630 for various processes (such as the process described above with reference to FIG. 1 and will not be described herein) to acquire keywords of chief complaint in a current medical record and to calculate a set of medical record-theme relevancy vectors. In another exemplary embodiment, storage medium 630 may store chief complaint in a standard medical record which may be stored in storage medium 630 in a standard case database or other existing or potential forms in the art or in future. In such an embodiment, CPU 622 may acquire chief complaint in a standard medical record from storage medium 630 (eg, a standard case database) for various processing (as described above with respect to FIG. 1 and will not be described herein) so as to acquire chief complaint in a medical record and calculate a set of vectors for standard medical record-theme relevancy. Storage medium 630 can also store various instructions for CPU 622 to perform the instructions described herein and/or other operations.

System 600 also comprises one or more wired or wireless network interfaces 650. The system 600 can remotely acquire chief complaint in a medical record and/or a standard medical record of a medical record subject via the network interface 650. For example, the medical record subject can provide chief complaint from a location remote from the system 600, such as the home or workplace of the medical record subject, a clinic in a remote town, etc., to implement remotely intelligent auxiliary diagnosis.

System 600 can also comprise one or more input and output interfaces 658, one or more keyboards 656, and/or one or more microphones (not shown).

The input and output interface can be, for example, a touch screen through which the medical record subject can interact with the system 600. For example, according to an exemplary embodiment, system 600 displays several symptoms, signs, and properties of various symptoms such as duration and severity through a display screen; the subject of the medical record selects the symptoms, signs and related properties he or she suffers on the touch screen display; the system 600 generates medical record complaint for the medical record subject after receiving various selections of the medical record subject for subsequent processing and calculation. According to another exemplary embodiment, the system 600 may rank the corresponding standard medical records from the highest to the lowest similarity value according to the level of each similarity value after determining the similarity between the current medical record complaint and the standard medical record complaint. That is, the standard medical records corresponding to the standard medical record complaint with high similarity of the current medical record is ranked first, and the standard medical record corresponding to the standard medical record complaint with low similarity of the current medical record is ranked next. And then the touch display screen shows the determined standard medical records sequentially to the medical record subject. Feedback to the questions of the standard medical records from the medical record subjects can be acquired to further assist in determining the target standard medical records.

In another embodiment, the medical record subject can provide his or her symptoms, signs, and related properties to the system 600 in text via the keyboard 656, and the system 600 generates a medical record complaint based on input from the medical record subject. In still another embodiment, the medical record subject provides its symptoms, signs, and related properties to the system 600 in the form of voice via a microphone or similar devices. The system 600 processes the voice record of the medical record subject and generates a machine-recognizable medical record complaint. Subsequent processing is generally performed.

The input/output interface 658 can also provide the current medical record complaint, the determined target standard medical record, and/or the relevancy between the two to the necessary personnel, such as a paramedic, a doctor, a pharmacist, a patient, a researcher, and the like.

System 600 can comprise one or more operating systems, such as Windows Server™, Mac OS X™, Unix™, Linux™, FreeBSD™, and the like.

Those skilled in the art understand that the system 600 can be in the form of a centralized layout or a distributed layout.

For example, for a large medical facility, system 600 can be placed in a centralized layout. In an exemplary embodiment of the system 600 in a centralized layout, a patient may go to a medical facility to provide his or her medical record at the front desk or other venues where an input and output device such as a touch display screen, a keyboard, a microphone, etc. may be provided for the patient to provide medical record complaint. Alternatively, staff can be arranged at the reception desk to receive the patient, organize the medical record complaint according to the patient's dictation and input it into the system 600. This is suitable for young or old patients, or patients who can not use the device to provide the medical record complaint. The system 600 then stores the medical record complaint and acquires keywords of the chief complaint in the current medical record by processing word segmentation, stopwords removal and keyword extraction to acquire keywords of the chief complaint in the current medical record. By comparing the current medical record theme relevancy vectors with the acquired or stored set of vectors for standard medical record-theme relevancy. And a standard medical record corresponding to the chief complaint in the current medical record is determined by calculating the similarity between the chief complaint in the current medical record and the chief complaint in the standard medical record.

System 600 can also further update standard medical records and LSI themes based on accumulated diagnostic results. The acquired standard medical records can be provided to paramedics so as to determine the diagnosis department and the doctor, and to the attending doctor or pharmacist for auxiliary treatment and prescription, and to the patient via the input and output device. The present invention is not limited in this respect.

For another example, a distributed system 600 may be provided in places where traffic is inconvenient or where people are not sparsely populated, such as remote towns, villages, and the like. CPU 622 and memory 632 of the system 600 can be provided in large medical institutions, while input and output interfaces, keyboards, microphones, and storage media can be provided in remote towns and villages. In some embodiments, a patient provide a medical record complaint via distributed input and output interfaces, a keyboard, a microphone, etc., The medical record complaint can be stored in the storage medium 630; and the stored medical record complaint can be provided to CPU 622 of the system 600 for calculation and diagnosis by the processor 622 via a wired or wireless network, or other physical means of transportation (the processing in the processor 622 is similar to the embodiment of the centralized layout, and will not be described herein).

It is understandable for those skilled in the art that the system 600 of the present invention may also take the form of a server/client (S/C). A patient can provide a medical record complaint via a fixed client set in a medical facility or via a mobile client set at home, office, etc.; the patient can also acquire the determined diagnosis result by the server from the client. And the present invention is not limited in this respect. A patient can also receive the questions in a standard medical record to be selected from the client and provide answers to these problems so as to assist in determining the target standard medical record; other personnel, such as paramedics, service personnel, medical personnel, researchers, etc., can also acquire a medical record complaint, a target standard medical record or related associations. The server can receive the medical record complaint from a fixed or mobile client, and perform process such as word segmentation, extraction, vector calculation, relevancy calculation so as to determine a target standard medical record corresponding to the medical record complaint; the server can also provide the determined target standard medical record to the client.

It is understandable for those skilled in the art that the system 600 of the present invention can also use cloud computing and/or cloud storage technologies to extract keywords in a medical record complaint, to calculate the set of medical record-theme relevancy vectors, to calculate similarity, to store medical record complaint and to build a standard medical record bank, etc. The present invention is not limited in this respect.

Through the description of the above embodiments, those skilled in the art can clearly understand that the present invention can be implemented by means of software plus necessary general hardware, and of course, by dedicated hardware, CPU, memory, special elements and so on. In general, functions performed by a computer program can be easily achieved with the corresponding hardware, and the specific hardware structure used to implement the same function can also be various, such as analog circuits, digital circuits, or dedicated circuits, etc. However, for the present invention, implementation by software program is preferred in most cases. Based on such understanding, the technical solution of the present invention essential or contributive to the prior art, may be expressed in the form of software products. Wherein the software products are stored in a readable storage medium, such as a floppy disk, USB, HDD, read-only memory (ROM, Read-Only Memory), random access memory (RAM, Random Access Memory), disk or CD, etc., including a number of instructions to make a computer device (can be a PC, a server, or network device, etc.,) implement the methods described in various embodiments of the present invention.

Finally, it is to be noted that the foregoing embodiments are merely for describing the technical solutions of the present invention and not intended to limit the present invention. Although the present invention has been described in details by the foregoing embodiments, it should be understood by those skilled in the art that modifications may be made to the technical solutions mentioned in the foregoing embodiment, or equivalent replacements may be made to part of the technical features, and these modifications or replacements shall fall into the spirit and scope of the technical solutions of the embodiments of the present invention.

What is claimed is:

1. An intelligent auxiliary diagnosis method performed by a computer, comprising:
calculating relevancy between keywords of chief complaint in a current medical record and Latent Semantic Indexing (LSI) themes to determine a set of vectors for current medical record-theme relevancy;
calculating relevancy between keywords of chief complaint in a standard medical record and the LSI themes to determine a set of vectors for standard medical record-theme relevancy;
calculating similarity between the chief complaint in the current medical record and the chief complaint in the standard medical record based on the set of vectors for current medical record-theme relevancy and the set of vectors for standard medical record-theme relevancy; and
determining a standard medical record according to the similarity.

2. The method of claim 1, further comprising:
ranking the determined similarity based on a plurality sets of vectors for standard medical record-theme relevancy; and
determining a target standard medical record according to a result of ranking and feedback information based on the standard medical record.

3. The method of claim 2, wherein the step of determining a target standard medical record, according to a result of ranking and feedback information based on the standard medical record, further comprises:
comparing ordered standard questions in each standard medical record with the feedback information based on the standard medical record starting from a standard medical record with the highest similarity; and
replacing a plurality of standard medical records in sequence based on comparison of relevancy until the comparison of ordered standard questions in the plurality of standard medical records are completed.

4. The method of claim 3, wherein the step of replacing the plurality of standard medical records in sequence based on the comparison of relevancy until the comparison of ordered standard questions in the plurality of standard medical records are completed further comprises:
selecting ordered standard questions in the next standard medical record in sequence, if comparison of the ordered standard questions in each of the standard medical records with the feedback information based on the standard medical record fails to meet a set standard.

5. The method of claim 2, wherein the feedback information based on the standard medical record is answer information acquired from a patient, answer information of the current medical record feedback or answer information of historical medical record feedback.

6. The method of claim 3, wherein the plurality of standard medical records correspond to a standard medical record database; wherein the standard medical record database comprises a bank of standard medical record chief complaint, a bank of ordered standard question, and a bank of standard answer corresponding to the ordered standard question bank.

7. The method of claim 1, wherein before the step of calculating relevancy between keywords of chief complaint in a current medical record and LSI themes to acquire a set of vectors for current medical record-theme relevancy, the method further comprises:
acquiring the chief complaint in the current medical record and performing word segmentation, stopwords removal and keyword extraction on the chief complaint in the current medical record to acquire the keywords of the chief complaint in the current medical record.

8. The method of claim 1, wherein a process of acquiring the LSI themes comprises:
performing word segmentation and stopwords removal on the chief complaint in the standard medical record to acquire a plurality of words; and
classification operating the plurality of words to acquire a plurality of LSI themes, according to the frequency of each of the words appearing in the chief complaint in the standard medical record.

9. The method of claim 8, wherein the step of classification operating the plurality of words to acquire the plurality of LSI themes, according to the frequency of each of the words appearing in the chief complaint in the standard medical record comprises:
numbering the words according to the serial numbers of the words in a medical dictionary and calculating the frequency of the words appearing in the chief complaint in the standard medical record; constructing a standard medical record chief complaint document vector containing a pair of the number and the frequency as an element; and
calculating TF-IDF value of the word corresponding to each element in the standard medical record chief complaint document vector to acquire a TF-IDF vector, and acquiring an LSI model by the TF-IDF vector training to set the LSI themes.

10. An intelligent auxiliary diagnosis system, comprising:
one or more non-volatile memories; and
a processor, wherein the processor comprises:
a first relevancy calculation module configured to calculate relevancy between keywords of chief complaint in a current medical record and Latent Semantic Indexing (LSI) themes to determine a set of vectors for current medical record-theme relevancy;
a second relevancy calculation module configured to calculate relevancy between keywords of chief complaint in a standard medical record and the LSI themes to determine a set of vectors for standard medical record-theme relevancy;
a similarity calculation module configured to calculate the similarity between the chief complaint in the current medical record and the chief complaint in the standard medical record, based on the set of vectors for current medical record-theme relevancy and the set of vectors for standard medical record-theme relevancy; and
a medical record determination module configured to determine a corresponding standard medical record according to the similarity.

11. A machine-readable storage medium, wherein the machine-readable storage medium stores machine executable instructions; the machine executable instructions are configured to enable a machine to execute the steps below:
calculating relevancy between keywords of chief complaint in a current medical record and Latent Semantic Indexing (LSI) themes to determine a set of vectors for current medical record-theme relevancy;
calculating relevancy between keywords of chief complaint in a standard medical record and the LSI themes to determine a set of vectors for standard medical record-theme relevancy;
calculating, the similarity between the chief complaint in the current medical record and the chief complaint in the standard medical record based on the set of vectors for current medical record-theme relevancy and the set of vectors for standard medical record-theme relevancy;
determining a corresponding standard medical record according to the similarity.
